(19) United States
(12) Patent Application Publication
(10) Pub. No.: US 2015/0064143 A1
(43) Pub. Date: Mar. 5, 2015

(54) IONICALLY CROSS-LINKABLE ALGINATE-GRAFTED HYALURONATE COMPOUND

(71) Applicant: INDUSTRY-UNIVERSITY COOPERATION FOUNDATION HANYANG UNIVERSITY, SEOUL (KR)

(72) Inventors: Kuen Yong LEE, Seoul (KR); Hong Hyun PARK, Seoul (KR)

(21) Appl. No.: 14/476,726

(22) Filed: Sep. 3, 2014

(30) Foreign Application Priority Data
Sep. 4, 2013 (KR) 10-2013-0106333
Aug. 29, 2014 (KR) 10-2014-0113971

(51) Int. Cl. A61K 31/738; A61L 27/36; A61K 35/12; A61L 27/20; C08B 37/08; A61K 35/32

(52) U.S. Cl. CPC A61K 31/738; C08B 37/0072; A61K 35/32; A61K 35/12; A61L 27/20; A61L 27/3612; A61L 27/3654; A61L 2430/06
USPC 424/93.7; 536/3; 530/331; 514/54

(57) ABSTRACT

The present invention provides an ionically cross-linkable alginate-grafted hyaluronate compound containing alginate and hyaluronate, the alginate and the hyaluronate forming a covalent linkage. The use of the compound can easily provide a cross-linked hyaluronate by adding a divalent cation without using a chemical cross-linking agent. The present invention also provides a method for cell transplantation that includes preparing a composition comprising the compound of claim 1 and cells for transplantation, and administering to a subject in need thereof the composition.

### CROSS-REFERENCE TO RELATED APPLICATION

This application claims priority to and the benefit of Korean Patent Application No. 10-2013-0106333, filed on Sep. 4, 2013, and No. 10-2014-0113971, filed on Aug. 29, 2014, the disclosure of which is incorporated herein by reference in its entirety.

### BACKGROUND

1. Field of the Invention

The present invention relates to an alginate-grafted hyaluronate compound.

2. Background of Technique

Natural polymer-based hydrogels have been used in various biomedical applications, due to their superior intrinsic biocompatibility compared to synthetic polymer-based hydrogels. Natural polymers have relatively low potential for toxicity, which is crucial with regard to biomaterial design. Among natural polymers, hyaluronate has been used in many biomedical applications, including drug delivery [1,2] and tissue engineering [3,4] applications, due to excellent biocompatibility and biological functionality. This polysaccharide is composed of repeating units of β-1,4-D-glucuronic acid-β-1,3-N-acetyl-D-glucosamine residues [5], and is abundant in synovial fluid and extracellular matrix. Hydrogels prepared from hyaluronate are particularly attractive, due to their excellent biological function, excellent viscoelastic properties, high water content and biodegradable properties. One typical preparation method of hyaluronate gels is chemical cross-linking [6-9]. Chemical cross-linking reagents, however, can induce acute or chronic side effects, such as immune and inflammatory responses[10]. This may cause potential risk, and limit wide biomedical applications of hyaluronate.

Physical cross-linking is an alternative approach that can be applied in overcoming toxicity issues brought about by chemical cross-linking. Hydrogels can be physically prepared, by varying external environments around stimulus-responsive polymers (e.g., temperature[11,12] and/or pH[13,14]), or by inducing physical interactions (e.g., ionic[15,16] or hydrophobic interactions[17,18]) to polymers. Alginate forms physically cross-linked structures in the presence of divalent cations (e.g., Ca²⁺), but in the absence of chemical cross-linkers. Alginate is a linear copolymer composed of blocks of β-D-mannuronate (M) and α-L-guluronate (G) residues [19,20]; it is also a naturally derived biomaterial. It is utilized in biomedical applications owing to its excellent biocompatibility and low toxicity [21,22]. Specifically, alginate modified with cell-adhesive motifs (e.g., RGD peptide) has often been utilized as injectable hydrogels for tissue engineering applications [23,24].

Articular cartilage is important for overall individual well-being. Articular cartilage functions and structures often get interrupted or damaged, due to physical injuries or degenerative diseases, such as osteoarthritis. Because cartilage tissue is both a neural and a vascular, chondrocyte proliferation is slow, and tissue defects are rarely spontaneously recovered[25,26]. Tissue engineering approaches using hydrogels have demonstrated great potential for treating articular cartilage defects through minimally-invasive chondrocyte delivery. Such minimally-invasive chondrocyte delivery may reduce cost, recovery time, and patient pain [27]. Another benefit of hydrogels is their superior viscoelastic properties. Viscoelastic properties of hydrogels are similar to those of cartilage natural extracellular matrix (ECM) materials [28-30]. Hyaluronate is a promising candidate for cartilage regeneration, as it is a main component of the ECM of native cartilage. In addition, hyaluronate interacts with CD44 on the chondrocyte surface, which is a molecule that is important in chondrocyte metabolism [31-33]. Hyaluronate has been extensively used in cartilage regeneration applications [34-36].

Throughout this application, various publications and patents are referred and citations are provided in parentheses. The disclosures of these publications and patents in their entities are hereby incorporated by references into this application in order to fully describe this invention and the state of the art to which this invention pertains.

### SUMMARY

The present inventors have endeavored to develop a method for easily cross-linking hyaluronate, which is a natural polymer, without a chemical cross-linking reagent. As a result, the present inventors have established that alginate-grafted hyaluronate which can be easily cross-linked through only addition of a divalent cation without using an excipient chemical cross-linking reagent can be prepared by grafting alginate to hyaluronate using a covalent linkage, and then completed the present invention.

Therefore, an aspect of the present invention is to provide an ionically cross-linkable alginate-grafted hyaluronate compound.

Another aspect of the present invention is to provide a composition for cell transplantation.

Still another aspect of the present invention is to provide a pharmaceutical composition for cartilage regeneration.

Still another aspect of the present invention is to provide a pharmaceutical composition for preventing or treating cartilage-injury disorders or diseases.

Still another aspect of the present invention is to provide a method for preparing an ionically cross-linkable alginate-grafted hyaluronate modification.

Other objects and advantages of the present invention will become apparent from the following detailed description together with the appended claims and drawings.

### BRIEF DESCRIPTION OF THE DRAWINGS

FIG. 1 represents schematic description for hyaluronate-g-alginate (HGA) and its hydrogel formation in the presence of calcium ions. Hyaluronate (HA) was initially modified with ethylenediamine (NH₂-HA). Then, NH₂-HA was reacted with alginate (AL) via carbodiimide chemistry.

FIG. 2a represents images of hyaluronate (HA) or hyaluronate-g-alginate (HGA) solution after mixing, in the presence (+) or absence (−) of calcium ions.

FIG. 2b represents image of HGA gels after injection via syringe needle.

FIG. 2c represents changes in storage modulus (G′, filled symbols) and loss modulus (G″, open symbols) of HGA1 (circles) and HA (squares) solutions in the presence of calcium ions.

FIG. 2d represents changes in storage modulus depending on hyaluronate content in hyaluronate-g-alginate gels ([HGA1]=2 wt %).

FIG. 2e represents changes in storage modulus depending on calcium concentration in hyaluronate-g-alginate gels ([HGA1]=2 wt %).

FIG. 2f represents gelation time of HGA gels.

FIG. 2g represents storage modulus of HGA gels depending on alginate molecular weight ([HGA1]=2 wt %, [CaSO₄]=29.7 Mm).

FIG. 3a represents changes in dry weight of either hyaluronate/alginate mixed gels or hyaluronate-g-alginate gels after treated with hyaluronidase for 2 weeks at 37° C.

FIG. 3b represents cross-sectional SEM images of alginate/hyaluronate mixed gels after incubation with hyaluronidase over 2 weeks at 37° C. ([polymer]=2 wt %, hyaluronate/alginate (mg/mg)=1, [Ca²⁺]=29.7 mM, [hyaluronidase]=100 μg/ml).

FIG. 3c represents cross-sectional SEM image of hyaluronate-g-alginate gels after incubation with hyaluronidase over 2 weeks at 37° C. ([polymer]=2 wt %, hyaluronate/alginate (mg/mg)=1, [Ca²⁺]=29.7 mM, [hyaluronidase]=100 μg/ml).

FIG. 4 represents histological images of tissue section stained with Alcian blue (a-d) and Sirius red (e-h). Immunohistochemical analysis of Matrillin-1 protein (i-l) and S100 protein (m-p) was also carried out. Tissue sections were retrieved from mice after six weeks of transplantation with primary chondrocytes ([cell]=1.0×10⁷ cells/ml) and hyaluronate-g-alginate gels.

FIG. 5a represents quantitative analysis of (a) sulfated GAG formation (**P<0.01).

FIG. 5b represents quantitative analysis of gene expression levels of SOX-9 (*P<0.05).

FIG. 5c represents quantitative analysis of gene expression levels of aggrecan (**P<0.01).

FIG. 5d represents quantitative analysis of gene expression levels of type II collagen in regenerated cartilage tissues (*p<0.05).

FIG. 6a represents ¹H-NMR spectra of hyaluronate, alginate, and hyaluronate-g-alginate.

FIG. 6b represents DMMB assay results for quantitative grafting efficiency of hyaluronate-g-alginate.

### DETAILED DESCRIPTION

In one aspect of the present invention, there is provided an ionically cross-linkable alginate-grafted hyaluronate compound containing alginate and hyaluronate, the alginate and the hyaluronate forming a covalent linkage.

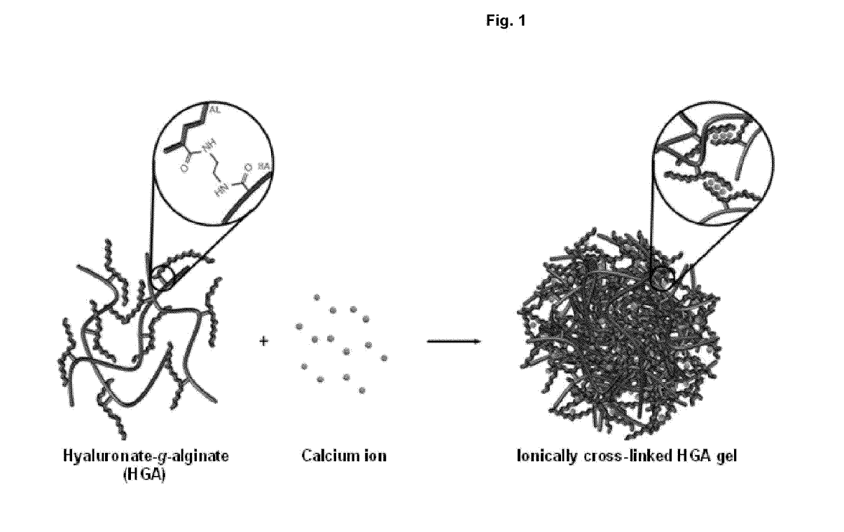

The present inventors have endeavored to develop a method for easily cross-linking hyaluronate, which is a natural polymer, without using a chemical cross-linking agent. As a result, the present inventors have established that alginate-grafted hyaluronate which can be easily cross-linked only through the addition of a divalent cation without using a chemical cross-linking reagent can be prepared by grafting alginate to hyaluronate using a covalent linkage (see FIG. 1).

As used herein, the term “alginate-grafted hyaluronate compound” refers to hyaluronate linked to alginate through a covalent linkage. As used herein, the term “alginate-grafted hyaluronate compound” may be exchangeably used with “alginate-grafted hyaluronate”, “hyaluronate-g-alginate”, “modified hyaluronate”, or “modified compound”, all of which have the same meaning. The alginate-grafted hyaluronate compound of the present invention can be used to easily and conveniently prepare a hydrogel via an ionic cross-linking. For the formation of a hyaluronate hydrogel in the prior art, a method of forming a hydrogel using a chemical cross-linking reagent has been employed. However, the alginate-grafted hyaluronate compound of the present invention can easily induce an ionic cross-linking reaction by addition of divalent cations, such as Ca²⁺, Ba²⁺, Cu²⁺, Fe²⁺, and Mg²⁺, due to cross-linking characteristics of alginate introduced into hyaluronate. This is important in that the possibility that the chemical cross-linking reagent may cause side effects, such as an immune or inflammation response, in the body can be eliminated. Therefore, a divalent cation, preferably Ca²⁺, may be added to the alginate-grafted hyaluronate compound before the injection of the compound into the body, and then the compound may be injected to the body before gelation. After the injection, the compound can form a hydrogel through prompt gelation behavior. In addition, a hydrogel in which alginate and hyaluronate are uniformly dispersed all over can be formed.

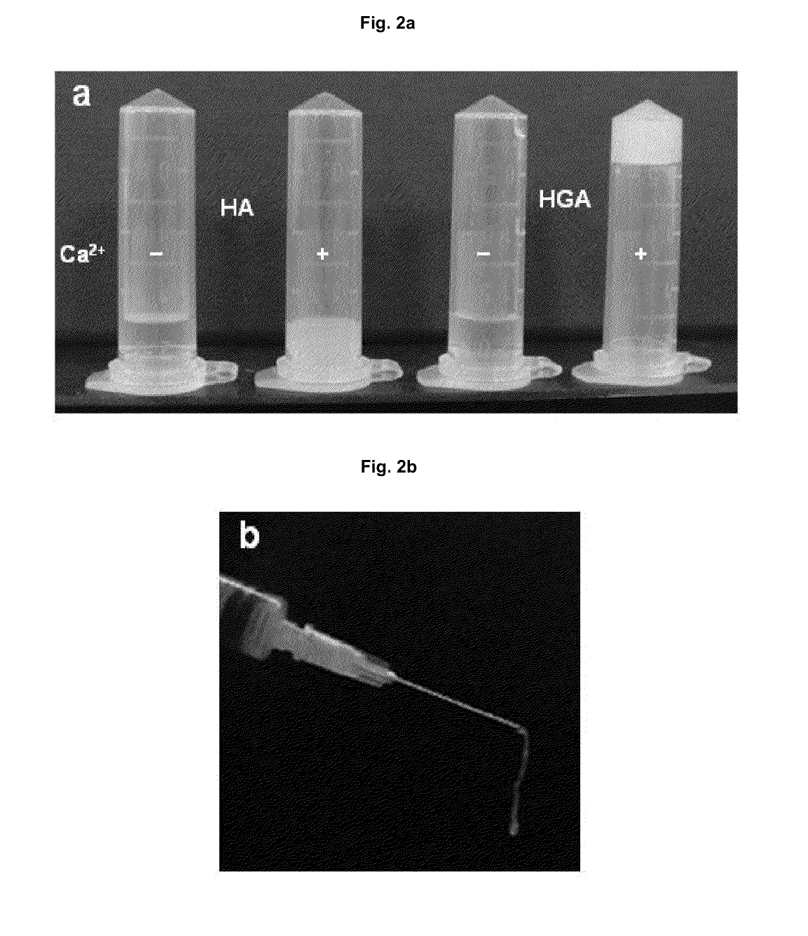

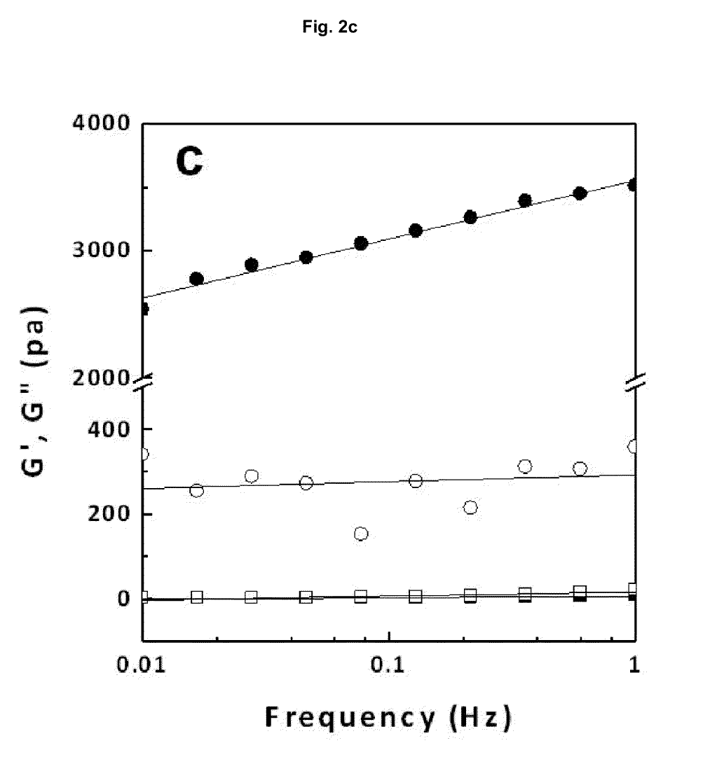

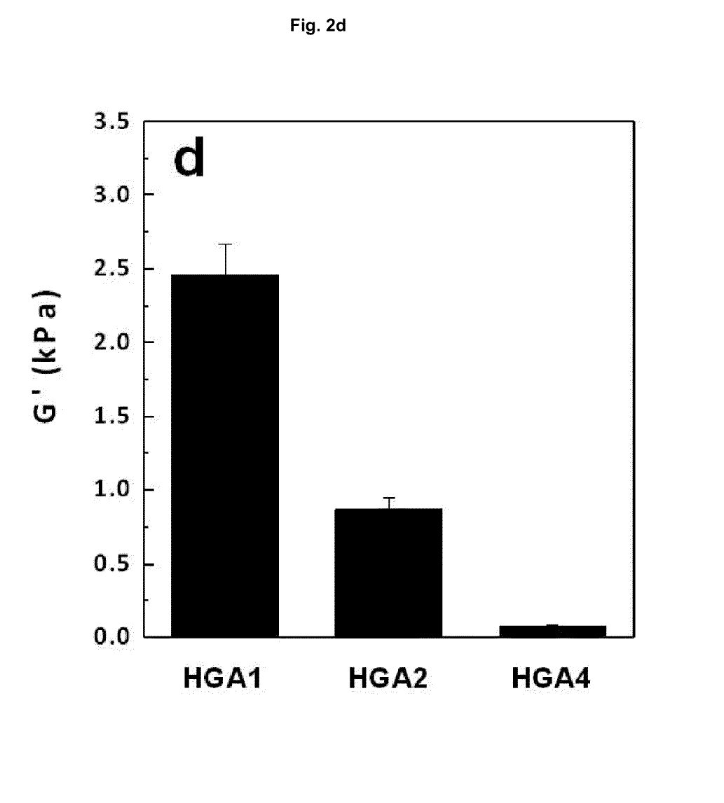

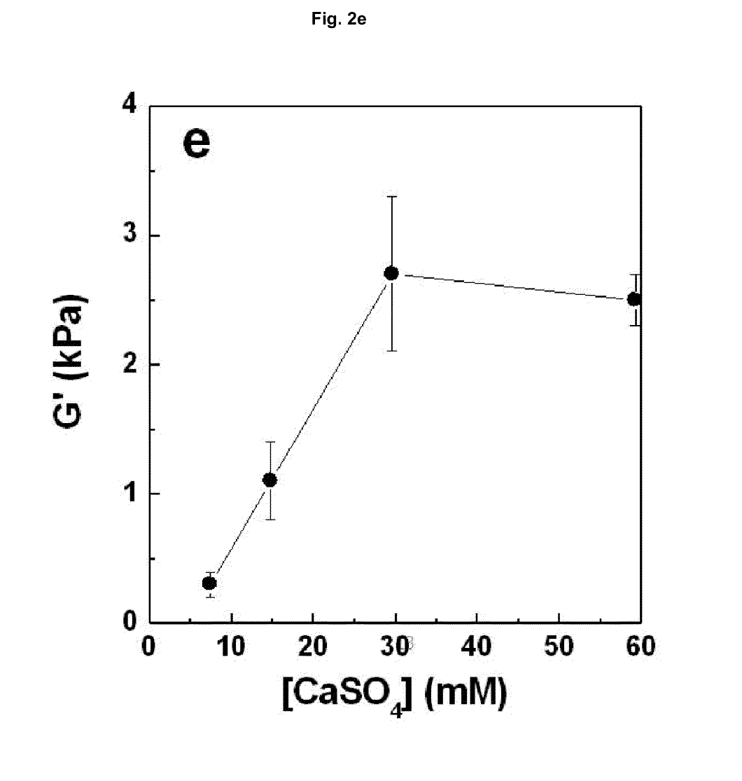

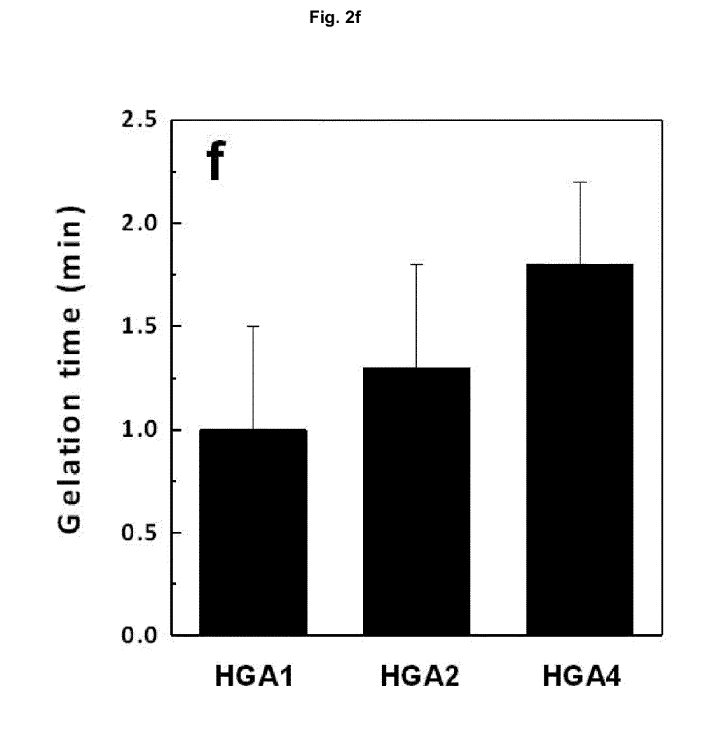

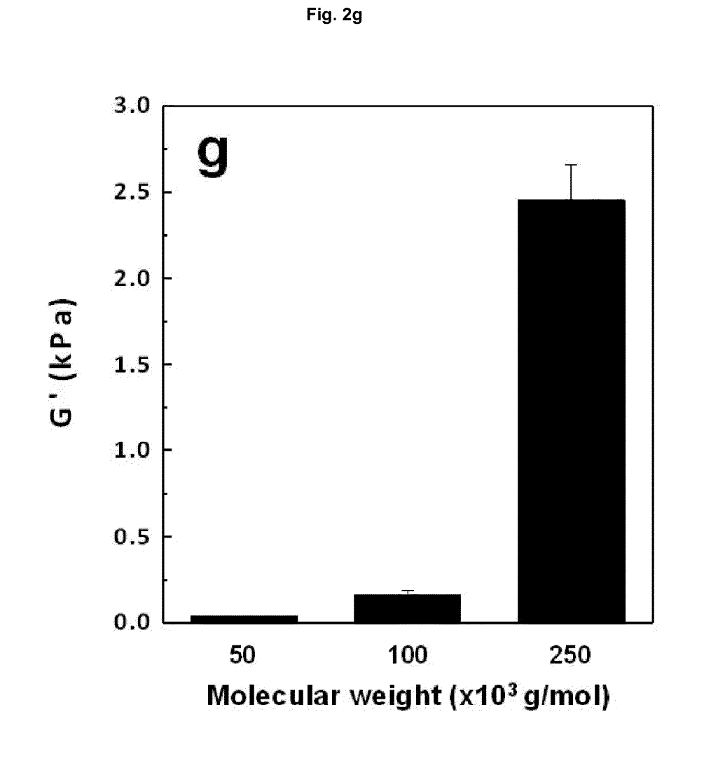

A hydrogel composition prepared from the modified compound of the present invention can control mechanical properties and gelation behavior according to the degree of introduction of alginate. The present inventors have verified that as the weight ratio of alginate increases from 1:4 (hyaluronate:alginate) to 1:1, the elastic coefficient of the ionic cross-linking hydrogel also increases (see FIG. 2d). The reason seems to be that the number of ionically cross-linkable points increases on the structure. The mechanical properties of the ionically cross-linked alginate-grafted hyaluronate hydrogel are shown to have lower levels than the ionically cross-linked alginate gel, but are sufficient to maintain a gel structure in the body after transplantation. In addition, the hydrogel composition prepared from the modified compound control mechanical properties and gelation behavior according to the amount of divalent cations added.

In addition, the alginate-grafted hyaluronate compound of the present invention has many advantages compared with an alginate/hyaluronate mixture. The modified hyaluronate enables the formation of a hydrogel in which alginate and hyaluronate are homogeneously distributed; have a significantly shortened gelation time and thus can be transplanted in the body immediately after the addition of a divalent cation for gelating the modified hyaluronate; and can be gelated in a short time even after the transplantation into the body and thus very useful in application fields including cell transplantation and the like. For example, in the case where chondrocytes are transplanted into the part of articulation, a transplanted subject can behave in a short time without requiring a long waiting time even after the cell transplantation. Further, the modified hyaluronate can be gelated using a divalent cation based on characteristics of introduced alginate, without using a chemical cross-linking reagent, and thus has superior biocompatibility, thereby avoiding various side effects due to the use of the chemical cross-linking agent. Further, the content ratio between alginate and hyaluronate, the content of divalent cations, and the like are controlled, thereby facilitating the control of mechanical properties.

According to an embodiment of the present invention, the preferable molecular weight of alginate, which is usable to prepare the alginate-grafted hyaluronate compound of the present invention, is 10,000-1,000,000 g/mol, and the preferable molecular weight of hyaluronate is 10,000-1,000,000 g/mol.

According to an embodiment of the present invention, the weight ratio between alginate and hyaluronate contained in the alginate-grafted hyaluronate compound of the present invention is 10:1 to 1:10. The weight ratio between alginate and hyaluronate is specifically 5:1 to 1:8, more specifically 2:1 to 1:6, more specifically 1:1 to 1:4, more specifically 1:1.2 to 1:3, still more specifically 1:1.5 to 1:2.5.

According to an embodiment of the present invention, the covalent linkage between alginate and hyaluronate is achieved via a linker capable of forming a covalent linkage between a carboxyl group of alginate and a carboxyl group of hyaluronate. As the linker forming a covalent linkage between hyaluronate and alginate, any linker that has at least two functional groups capable of reacting with carboxyl groups to form a covalent linkage and is known in the art can be used without limitation.

According to an embodiment of the present invention, the linker of the present invention which links alginate and hyaluronate through a covalent linkage is selected from diamine, divinylsulfone, 1,4-butanediol diglycidyl ether (BDDE), glutaraldehyde, carbodiimide, hydroxysuccinimide, imidoester, maleimide, haloacetyl, disulfide hydroazide, and alkoxy amine. According to another embodiment of the present invention, the linker is diamine, and preferably ethylene diamine.

In order to link alginate and hyaluronate through a covalent linkage by using the linker, any one selected from alginate and hyaluronate is preferentially treated, and here, it is important to use a large amount of linker to prevent self-assembling. As used herein, the term “self-assembling” refers to the linkage of alginate molecules or hyaluronate molecules, that is, homologous molecules, via one linker, but not the linkage of alginate and hyaluronate molecules, that is, heterogeneous molecules via one linker, and includes the linkage of several carboxyl groups in one molecule with one linker.

As used herein, alginate or hyaluronate which first reacts with a linker is called a “first reactant”, and a compound which is later added in order to be linked to the linker linked to the first reactant is called a “second reactant”. That is, when the first reactant is alginate, the second reactant is hyaluronate, and when the first reactant is hyaluronate, the second reactant is alginate. Before the first reactant linked to the linker reacts with the second reactant, a linker that is not linked to the first reactant is removed by filtration, for example, a dialysis procedure. After that, through additional reaction with hyaluronate or alginate, which is a second reactant, an alginate-grafted hyaluronate compound is finally obtained.

According to an embodiment of the present invention, a cell adhesive peptide is linked to at least one of alginate and hyaluronate of the present invention. Any one cell adhesive peptide that is known in the art may be used.

According to an embodiment of the present invention, the cell-adhesive peptide of the present invention is at least one selected from the group consisting of RGD(Arg-Gly-Asp), RGDS(Arg-Gly-Asp-Ser) (SEQ ID NO: 1), RGDC(Arg-Gly-Asp-Cys) (SEQ ID NO: 2), RGDV(Arg-Gly-Asp-Val) (SEQ ID NO: 3), RGES(Arg-Gly-Glu-Ser) (SEQ ID NO: 4), RGDSPASSKP(Arg-Gly-Asp-Ser-Pro-Ala-Ser-Ser-Lys-Pro) (SEQ ID NO: 5), GRGDS(Gly-Arg-Gly-Asp-Ser) (SEQ ID NO: 6), GRADSP(Gly-Arg-Ala-Asp-Ser-Pro) (SEQ ID NO: 7), KGDS(Lys-Gly-Asp-Ser) (SEQ ID NO: 8), GRGDSP(Gly-Arg-Gly-Asp-Ser-Pro) (SEQ ID NO: 9), GRGDTP(Gly-Arg-Gly-Asp-Thr-Pro) (SEQ ID NO: 10), GRGES(Gly-Arg-Gly-Glu-Ser) (SEQ ID NO: 11), GRGDSPC(Gly-Arg-Gly-Asp-Ser-Pro-Cys) (SEQ ID NO: 12), GRGESP(Gly-Arg-Gly-Glu-Ser-Pro) (SEQ ID NO: 13), SDGR(Ser-Asp-Gly-Arg) (SEQ ID NO: 14), YRGDS(Tyr-Arg-Gly-Asp-Ser) (SEQ ID NO: 15), GQQHHLGGAKQAGDV (Gly-Gln-Gln-His-His-Leu-Gly-Gly-Ala-Lys-Gln-Ala-Gly-Asp-Val) (SEQ ID NO: 16), GPR(Gly-Pro-Arg), GHK(Gly-His-Lys), YIGSR(Tyr-Ile-Gly-Ser-Arg) (SEQ ID NO: 17), PDSGR(Pro-Asp-Ser-Gly-Arg) (SEQ ID NO: 18), CDPGYIGSR(Cys-Asp-Pro-Gly-Tyr-Ile-Gly-Ser-Arg) (SEQ ID NO: 19), LCFR(Leu-Cys-Phe-Arg) (SEQ ID NO: 20), EIL(Glu-Ile-Leu), EILDV(Glu-Ile-Leu-Asp-Val) (SEQ ID NO: 21), EILDVPST(Glu-Ile-Leu-Asp-Val-Pro-Ser-Thr) (SEQ ID NO: 22), EILEVPST(Glu-Ile-Leu-Glu-Val-Pro-Ser-Thr) (SEQ ID NO: 23), LDV(Leu-Asp-Val), and LDVPS(Leu-Asp-Val-Pro-Ser) (SEQ ID NO: 24). More specifically, RGD(Arg-Gly-Asp) may be used. The cell adhesive peptide serves to improve an interaction between cells and a hydrogel formed by the modified hyaluronate.

In another aspect of the present invention, there is provided a method for cell transplantation, comprising:

(a) preparing a composition comprising the alginate-grafted hyaluronate compound and cells for transplantation; and

(b) administering to a subject in need thereof the composition of step (a).

The alginate-grafted hyaluronate compound may be provided as a biocompatible support for cells for transplantation by forming a hydrogel in the body. As used herein, the term “composition” of step (a) refers to a composition which serves as a support for cells in need of transplantation into the body and thus can be injected into the body together with cells at the time of cell transplantation. The kind of cells as an object of cell transplantation is not particularly limited. The composition for cell transplantation may be mixed with cells for transplantation, and then injected into a desired site using for example a syringe. When the composition of the present invention mixed with cells is mixed with a divalent cation before being injected into the body, the injected composition may promptly form a hydrogel at the transplantation site, so that the cells can be transplanted into the body. The cells for transplantation may be contained at 1×10⁵ to 5×10⁸ cells/ml within 0.1 wt % to 10 wt % of a polymer solution for the composition for cell transplantation.

The composition of step (a) of the present invention may be used for transplantation of various stem cells. The kind of stem cell to which the present invention is applied is not limited, and the present invention may be applied to cells that have characteristics of stem cells, that is, undifferentiation, indefinite proliferation, and differentiation ability into particular cells. The stem cells to which the present invention is applied are largely classified into two: pluripotent stem cells including embryonic stem cells (ES) and embryonic germ cells (EG) and multipotent stem cells. The embryonic stem cells are derived from inner cell mass (ICM) of the blastocyst, and the embryonic germ cells are derived from primordial germ cells present in 5-10 week aged gonadal ridge. Meanwhile, the multipotent stem cells are found in embryonic tissues, fetus tissues, or adult tissues, and include adult stem cells. The pluripotent stem cells are indefinitely proliferated in vitro and have the capability to differentiate into various cells derived from all three germ layers (ectoderm, mesoderm, and endoderm). Meanwhile, the multipotent stem cells have capability to differentiate into particular tissues derived therefrom, and their self-renewal potency is conventionally restricted to the lifetime of organisms. The source of multipotent stem cells includes all type of tissues, and is mainly isolated from, particularly, bone marrow, blood, liver, skin, intestine, spleen, brain, skeletal muscle, and dental pulp. In the case where the composition of the present invention is used in the transplantation of stem cells, the therapeutic composition of the present invention further includes a stem cell differentiation inducer. Preferable examples of the stem cell differentiation inducer include retinoic acid, ascorbic acid, melatonin, or several growth factors (e.g., glial cell line-derived neurotrophic factor (GDNF), epidermal growth factor (EGF), and nerve growth factor (NGF)). The composition for cell transplantation of the present invention has superior biocompatibility since a chemical cross-linking agent for forming a hydrogel is not used. In addition, the use of hydrogel containing a cell adhesive peptide can improve an interaction with cells and thus promote higher tissue regeneration efficiency.

According to an embodiment of the present invention, the cells for transplantation of the present invention are selected from the group consisting of chondrocytes, myoblasts, hepatocytes, osteoblasts, embryonic stem cells, embryonic germ cells, adult stem cells, mesenchymal stem cells, neural stem cells, endothelial stem cells, hematopoietic stem cells, liver stem cells, cardiac stem cells, pancreatic stem cells, endothelial progenitors, outgrowth endothelial cells, hematopoietic stem cells, human neural stem cells, satellite cells, intestinal epithelial cells, smooth muscle cells, and fibroblasts.

In another aspect of the present invention, the present invention provides a method for cartilage regeneration, comprising administrating to a subject in need thereof a pharmaceutical composition comprising a pharmaceutically effective amount of the alginate-grafted hyaluronate compound. The pharmaceutical composition for cartilage regeneration comprising a pharmaceutically effective amount of the alginate-grafted hyaluronate compound. The modified hyaluronate of the present invention forms a hydrogel in the body and thus acts as a support for primary chondrocytes, stem cells, or the like, thereby can be transplanted into the site in the body in need of cartilage regeneration. The pharmaceutical composition for cartilage regeneration of the present invention can be transplanted into the body together with cells for transplantation, through injection, and can be easily and promptly gelated after being transplanted. In the pharmaceutical composition of the present invention, the cells for transplantation may be contained at 1×10⁵ to 5×10⁸ cells/ml within 0.1 wt % to 10 wt % of a polymer solution for the composition for cell transplantation.

The pharmaceutical composition of the present invention may further include a pharmaceutically acceptable carrier. The pharmaceutically acceptable carrier included in the therapeutic agent composition of the present invention is conventionally used at the time of formulation of medicines, and examples thereof may include, but are not limited to, lactose, dextrose, sucrose, sorbitol, mannitol, starch, acacia gum, calcium phosphate, alginate, gelatin, calcium silicate, microcrystalline cellulose, polyvinyl pyrrolidone, cellulose, water, syrup, methyl cellulose, methyl hydroxybenzoate, propyl hydroxybenzoate, talc, magnesium stearate, and mineral oil. The therapeutic agent composition of the present invention may further include, in addition to the above components, a lubricant, a wetting agent, a sweetening agent, a flavoring agent, an emulsifier, a suspension, a preservative, and the like. Pharmaceutically acceptable carriers and agents are described in detail in Remington's Pharmaceutical Sciences (19th ed., 1995).

The pharmaceutical composition of the present invention may be parenterally administered, and local injection is the most preferable manner of administration.

The suitable dose of the composition of the present invention may vary depending on various factors, such as method of formulation, manner of administration, age, body weight, sex, and morbidity of the patient, diet, route of administration, excretion rate, and response sensitivity. Meanwhile, the dose of the composition of the present invention is 1×10⁵ to 5×10⁸ cells/ml per a single dose, and the dose may be adjusted as needed.

The composition of the present invention may be formulated into a unit dosage form or a multidose container, using a pharmaceutically acceptable carrier and/or excipient according to the method easily conducted by a person having ordinary skills in the art to which the present invention pertains. Here, the dosage form may be a solution in an oily or aqueous medium, a suspension, or an emulsion, and may further include a dispersant or a stabilizer.

The pharmaceutical composition of the present invention enables the remarkable formation of intercellular networking to sufficiently exert functions of the cells for transplantation, thereby actually exhibiting improved therapeutic efficacy in disease animals.

According to an embodiment of the present invention, the pharmaceutical composition of the present invention further includes primary chondrocytes or stem cells for cartilage regeneration. The pharmaceutical composition of the present invention serves as a support for primary chondrocytes or stem cells for cartilage regeneration, and remarkably increases the success rate of cell transplantation.

In another aspect of the present invention, there is provided a method for preventing or treating cartilage-injury disorders or diseases, comprising administrating to a subject in need thereof a pharmaceutical composition comprising a pharmaceutically effective amount of the alginate-grafted hyaluronate compound.

According to an embodiment of the present invention, the cartilage-injury disorders are selected from the group consisting of osteoarthritis, rheumatoid arthritis, meniscus Injury, costochondritis, relapsing polychondritis, and chondrosarcoma. The method for preventing or treating cartilage-injury disorders or diseases is associated with the method for cell transplantation and the method for cartilage regeneration, and descriptions of overlapping contents thereof are omitted to avoid excessive complication of the specification.

In another aspect of the present invention, there is provided a method for preparing an ionically cross-linkable alginate-grafted hyaluronate compound, the method including:

(a) reacting alginate or hyaluronate as a first reaction material with a linker to covalently link the linker to a carboxyl group of the alginate or the hyaluronate; and

(b) reacting the reaction product obtained in step (a) with hyaluronate or alginate as a second reactant to obtain an alginate-grafted hyaluronate compound in which the alginate is linked to the hyaluronate via the linker.

According to an embodiment of the present invention, the linker of step (a) is used at such an amount as to be linked to all the carboxyl groups of the alginate or hyaluronate as the first reactant.

The method for preparing an alginate-grafted hyaluronate compound of the present invention is associated with a method for preparing “an alginate-grafted hyaluronate compound” according to another aspect of the present invention, and thus descriptions of overlapping contents thereof are omitted to avoid excessive complication of the specification.

### EXAMPLES

### Materials and Methods

### Synthesis of Hyaluronate-g-Alginate

All alginate samples were originally modified with RGD peptides in order to enhance cellular interactions. Briefly, 1 g of sodium alginate (molecular weight=200,000-300,000; FMC Biopolymer) was dissolved in 100 ml of 0.1 M 2-(N-morpholino) ethanesulfonic acid (MES; Sigma-Aldrich) buffer solution (pH 6.5, 0.3 M NaCl). Then, 16.7 mg of RGD peptide with the sequence of (glycine)4-arginine-glycine-aspartic acid-serine-proline (G4RGDSP; Anigen) was added to the alginate solution in the presence of 1-ethyl-3-(dimethylaminopropyl) carbodiimide (EDC; Sigma-Aldrich) and N-hydroxysulfosuccinimide (Sulfo-NHS; Thermo), in conjunction with vigorous solution stirring. Reaction was allowed to take place for 20 h at room temperature. Resultant solution was dialyzed for four days (molecular weight cut off=3,500), and then treated with charcoal. After filtration with a 0.22-μm filter for sterilization, solution was frozen and lyophilized. Hyaluronate (molecular weight=600,000-850,000; Lifecore) was first reacted with ethylenediamine (Sigma-Aldrich), prior to conjugation with alginate. Synthesis of NH₂-hyaluronate was carried out with the same procedure with EDC and NHS, as described above. A 10-fold molar ratio excess of ethylenediamine was added to inhibit intermolecular cross-linking between hyaluronate chains during the reaction. The reaction was allowed to proceed for 20 h at room temperature. The solution was then dialyzed, treated with charcoal, filtered with a 0.22-μm filter for sterilization, and lyophilized. Alginate was coupled to NH₂-hyaluronate via carbodiimide chemistry according to the above-described procedure (FIG. 1).

### Nuclear Magnetic Resonance Spectroscopy

Hyaluronate, alginate and hyaluronate-g-alginate were analyzed by 1H nuclear magnetic resonance (NMR) spectroscopy (Bruker Avance500 MHz) at 70° C. Samples were dissolved in D2O at 3 mg/ml.

### Dimethyl Methylene Blue (DMMB) Assay

DMMB assays were performed to quantify alginate/hyaluronate content in hyaluronate-g-alginate. Briefly, 16 mg of DMMB was dissolved in 25 ml of ethanol and filtered with filter paper, and 100 ml of 1 M guanidine hydrochloride containing 0.17 M of sodium formate and 1 ml of formic acid was mixed with filtered DMMB. The solution was mixed with deionized water to a total volume of 500 ml. Each sample was diluted with deionized water, to yield a solution of 0.1 wt %. One milliliter of DMMB solution was added to 100 μl of each sample and mixed vigorously for 30 min. Incubated samples were centrifuged at 12,000 g for 10 min to precipitate the complex. Supernatant was removed and dried for 30 min at room temperature. Pellets were dissolved with 1 ml of decomplexation solution. Decomplexation solution was prepared with 50 mM of sodium acetate buffer (pH 6.8), containing 10% 1-propanol and 4 M guanidine hydrochloride. After 30 min of mixing, 100 μl of each sample was transferred to a 96-well plate. Absorbance was measured at 656 nm using a spectrophotometer (SpectraMax M2; Molecular Devices).

### Enzymatic Stability Test

Hyaluronate-g-alginate (HGA1) was dissolved in PBS and mixed with calcium sulfate to form gels ([polymer]=2 wt %, [CaSO₄]=29.7 mM) using two glass plates with a spacer (1 mm thickness). Gel disks were obtained using a punch (10 mm diameter) and placed into PBS solution containing hyaluronidase in the concentration range of 0 to 100 μg/ml, before being incubated at 37° C. for 24 h. Gel disks were frozen and lyophilized, before dry weight was measured. A physical mixture of hyaluronate and alginate was also used as a control ([polymer]=2 wt %, [CaSO₄]=29.7 mM). Cross-sectional images of the gels were observed after treatment with hyaluronidase by scanning electron microscopy (S-4800 UHR FE-SEM; Hitachi).

### Rheological Measurement

Viscoelastic properties of ionically cross-linked hyaluronate-g-alginate gels were measured using a rotational rheometer with a cone-and-plate (20 mm diameter plate, 4 degree cone angle) fixture (Bohlin Gemini 150; Malvern). A 150 m gap opening was set at the apex of the cone and plate, and operating temperature was set to be constant at 37±0.1° C.

### Cell Isolation and Culture

Primary chondrocytes were isolated from the articular cartilage of New Zealand white rabbits (four-week-old; Samtako). The rabbits were sacrificed, and cartilage tissue fragments were obtained from hind leg knee joints. After fragments were washed with cold PBS, they were minced and digested with 4.5 mg/ml collagenase type II (Worthington) in DMEM/F-12 containing 10% FBS and 1% penicillin-streptomycin over a 6-h period. Digested cell suspension was passed through a cell strainer (40 μm; SPL Life Science) to remove undigested tissue fragments. Cells were collected using a centrifuge, washed twice with PBS, and suspended in DMEM/F-12 media containing 10% FBS and 1% penicillin-streptomycin. Isolated cells were cultured using standard culture procedures, and cells corresponding to passage number 2 or below were used for in vivo transplantation.

### Cartilage Regeneration In Vivo

All animal procedures were performed in accordance with Hanyang University guidelines for the care and use of laboratory animals and were approved by the Institutional Animal Care and Use Committee (IACUC) of Hanyang University (HY-IACUC-12-060A). BALB/c nude mice (six-week-old; Nara Biotech) were anesthetized with intraperitoneal injections of Zoletil (Virbac)/Rompun (Bayer) solution (9:1). Primary chondrocytes (1.0×10⁷ cells/ml) were mixed with either RGD-alginate solution (AL) or RGD-alginate-grafted hyaluronate solution (HGA1, HGA2 or HGA4), before calcium sulfate slurries were mixed in ([polymer]=2 wt %). 100 μl of each cell-polymer mixture was subcutaneously injected into the dorsal region of the mouse (n=6 mice, per group). Six weeks after transplantation, mice were sacrificed, and the regenerated tissues were retrieved.

### Histological and Immunohistochemical Analysis

Retrieved tissue samples were washed with cold PBS, and volume was measured. Samples were transferred to 4% formaldehyde solution overnight for fixation, and then dehydrated by immersion in a series of increasing concentrations of ethanol solution. Following immersion into xylene and paraffin, samples were embedded with paraffin. The specimens were cross-sectioned to 5 μm thickness with a microtome (RM2145; Leica). Tissue sections were stained with Alcian blue or Sirius red. For visualization of Matrillin-1 protein expression, samples were treated with HRP-conjugated anti-Matrillin-1 primary antibody (1/100 diluted; Bioss) and visualized with a diaminobenzidine peroxidase substrate kit (Vector). Images were obtained using optical microscopy (Axioskop 40; Carl Zeiss). Tissue sections were treated with anti-S100 primary antibody (1/25 diluted; Abcam) and FITC-conjugated secondary antibody (1/100 diluted; Jackson) in a humid box at 4° C. to visualize S100 protein expression. The stained slide glass was mounted with VECTASHIELD® Mounting Medium with DAPI (Vector Laboratories). Fluorescent images were obtained by fluorescence microscopy (ECLIPSE TE2000-E; Nikon).

### Quantification of Sulfated Glycosaminoglycan (GAG)

For evaluation of GAG content in the regenerated tissues, a BlyscanTMsGAG assay kit (Biocolor) was used. Briefly, the retrieved tissues were lyophilized and weighted. The tissue fragments were put into 70 mM EDTA solution and incubated for 30 min at 37° C. to eliminate alginate from the tissues in order to exclude a potentiality of GAG detection from alginate in the hydrogels. After centrifugation, supernatant was removed. For extraction of GAGs, 500 μl of papain extraction solution (10 μl/ml) dissolved in 50 mM phosphate buffer (pH 6.5) containing 1 M NaCl, 5 mM cysteine HCl, and 1 mM EDTA was added to each sample and incubated at 60° C. overnight. A 1,9-dimethylmethylene blue reagent (1 ml) was then added, and samples were allowed to react over 30 min with gentle shaking. Following centrifugation at 10,000 rpm over 10 min, supernatant was drained to eliminate unbound dye, and the pellet was dried. A dissociation reagent was added to the sample pellet, and vigorous mixing took place. Spectrophotometer measurements were taken to observe samples and chondroitin 4-sulfate standards at 646 nm (SpectraMax M2; Molecular Devices).

### Gene Expression

Total RNA was isolated from retrieved tissues using an RNA isolation kit (RNA iso plus; Takara). Isolated RNA was reverse transcribed to cDNA using a reverse transcription master mix (ELPIS Biotech). Chondrogenic marker genes (SOX-9, aggrecan, and type II collagen) and housekeeping gene (GAPDH) expression was investigated by an ABI Prism 7500 real-time PCR system (Applied Biosystems), in conjunction with the use of SYBR® Premix Ex Taq™. The sequences of primers (IDT) were as follows: SOX-9, 5′-CTTCATGAAGATGACCGACGAG-3′ (SEQ ID NO: 25), 5′-CTCTTCGCTCTCCTTCTTGAGG-3′ (SEQ ID NO: 26); aggrecan, 5′-GTGAAAGGTGTTGTGTTCCACT-3′ (SEQ ID NO: 27), 5′-TGGGGTACCTGACAGTCTGAT-3′ (SEQ ID NO: 28); type II collagen, 5′-AAGAGCGGTGACTACTGGATAG-3′ (SEQ ID NO: 29), 5′-TGCTGTCTCCATAGCTGAAGT-3′ (SEQ ID NO: 30); GAPDH, 5′-GACATCAAGAAGGTGGTGAAGC-3′ (SEQ ID NO: 31), 5′-CTTCACAAAGTGGTCATTGAGG-3′ (SEQ ID NO: 32).

### Statistical Analysis

All data are presented as mean± standard deviation (n=6). Statistical analyses were performed using a Student's t-test. *P-values<0.05 and **P<0.01 were considered statistically significant.

### Results

### Characterization of Alginate-Grafted Hyaluronate

Hyaluronate-g-alginate (HGA) was designed and synthesized in the preparation of ionically cross-linkable hyaluronate. In brief, hyaluronate (HA) was first modified with ethylenediamine (NH₂-HA), followed by conjugation with alginate (AL) by carbodiimide chemistry (HA-g-AL; FIG. 1). Various alginate-g-hyaluronate samples were synthesized. Weight ratios of hyaluronate to alginate were 1, 2, and 4 (hereinafter referred to as HGA1, HGA2, and HGA4, respectively). RGD peptides were coupled to alginate to enhance cellular interactions of resultant HGA gels.

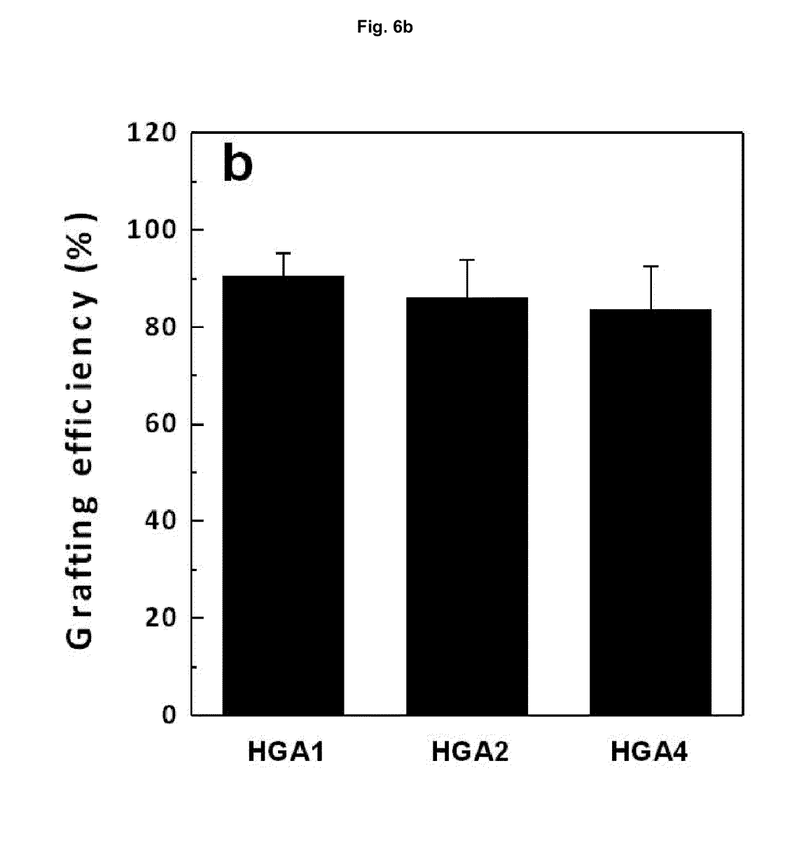

Hyaluronate-g-alginate synthesis was confirmed by ¹H-NMR spectroscopy (FIG. 6a). A peak area of δ=4.2-4.4 ppm increased after modification with ethylenediamine, indicating successful introduction of ethylenediamine to hyaluronate (NH₂-HA). Specific peaks of alginate and hyaluronate are concurrently shown on spectra at δ=4.2-4.7 and δ=2.5-2.6 (NCOCH₃), respectively, after NH₂-hyaluronate modification with alginate. Graft efficiency of HA-g-AL was determined by calculating the polymer content from the spectra (HGA1=93.7%, HGA2=82.6, HGA4=94.7%). Graft efficiency was also evaluated with dimethylmethylene blue (DMMB) assays (FIG. 6b). Hyaluronate does not bind to DMMB dye; thus, from this assay, only the alginate content in HA-g-AL was determined (HGA1=90.4±4.9%, HGA2=86.0±7.9%, HGA4=83.6±9.0%). The results found here were quite consistent with those obtained from ¹H-NMR spectroscopy.

### Hydrogel Formation of Hyaluronate-g-Alginate

Hydrogels were not formed upon mixture of hyaluronate solution with calcium ions ([hyaluronate]=2 wt %, [CaSO₄]=30 mM). In contrast, in the presence of calcium ions, hyaluronate-g-alginate was able to form gels ([HGA1]=2 wt %, [CaSO₄]=30 mM) (FIG. 2a). Most toxicity-related issues in hyaluronate-based hydrogels are derived from use of chemical cross-linkers, rather than stemming from issues related to the polymers[37,38]. Toxicity-related issues in typical hyaluronate gels, as caused by chemical cross-linking reagents after transplantation into the body, may be circumvented by use of ionically cross-linkable, hyaluronate-based hydrogels. Ionically cross-linkable alginate-g-Hyaluronate gels were able to be injected via a syringe with a 23-G needle (FIG. 2b). This is also attractive in terms of achieving minimally-invasive delivery of drugs and/or cells to the body.

### Characterization of Hyaluronate-g-Alginate Gels

Viscoelastic properties and gelation behaviors of hyaluronate-g-alginate gels were investigated through use of rotational rheometry (FIG. 2c-g). When HGA1 solution was mixed with calcium ions, the storage modulus (G′) was higher than the loss modulus (G″) at various frequencies (FIG. 2c), indicating that the mixture constructs a hydrogel structure via ionic cross-linking. In contrast, hyaluronate solutions did not form hydrogels upon calcium addition. We then investigated the effects of alginate content on the mechanical properties of ionically cross-linked hyaluronate-g-alginate gel (FIG. 2d). Hydrogels were prepared at constant polymer and calcium concentrations for all samples tested ([polymer]=2 wt %, [CaSO₄]=30 mM). Weight ratios between hyaluronate and alginate were varied. As weight ratio of hyaluronate-to-alginate increased from 1 to 4, gel mechanical properties changed significantly from 2.5±0.2 kPa to 0.075±0.001 kPa. We then monitored how changes in elastic properties of ionically cross-linked hydrogels depended on calcium concentrations (FIG. 2e). As calcium sulfate concentration increased from 7.4 mM to 30 mM, G′ values of hyaluronate-g-alginate gels increased from 0.3±0.1 kPa to 2.7±0.6 kPa. However, no further increase was observed when calcium sulfate concentration was increased to 60 mM. Gelation time of hyaluronate-g-alginate gels was not significantly influenced by the alginate content in the gels (FIG. 2f). Shear modulus of hyaluronate-g-alginate gels was dependent on alginate molecular weight (FIG. 2g). As alginate molecular weight increased from 50,000 to 250,000 g/mol, G′ of ionically cross-linked gels also increased from 0.04±0.003 to 2.4±0.2 kPa. Alginate-g-hyaluronate gels prepared with alginate of either 50,000 g/mol or 100,000 g/mol showed poor mechanical properties, even in the presence of calcium ions. It is very important to regulate the mechanical properties of polymer scaffold with regard to controlling cellular behaviors (e.g., adhesion, proliferation and migration) [39-41]. In addition, mechanical properties are also crucial in the modulation of stem cell differentiation, which has an immediate influence on tissue regeneration [41-44]. The mechanical properties of hyaluronate-g-alginate gels were easily adjustable through control of polymer composition and calcium ion concentration, which may be very attractive for use in tissue engineering.

### Enzymatic Stability of Alginate-g-Hyaluronate Gels

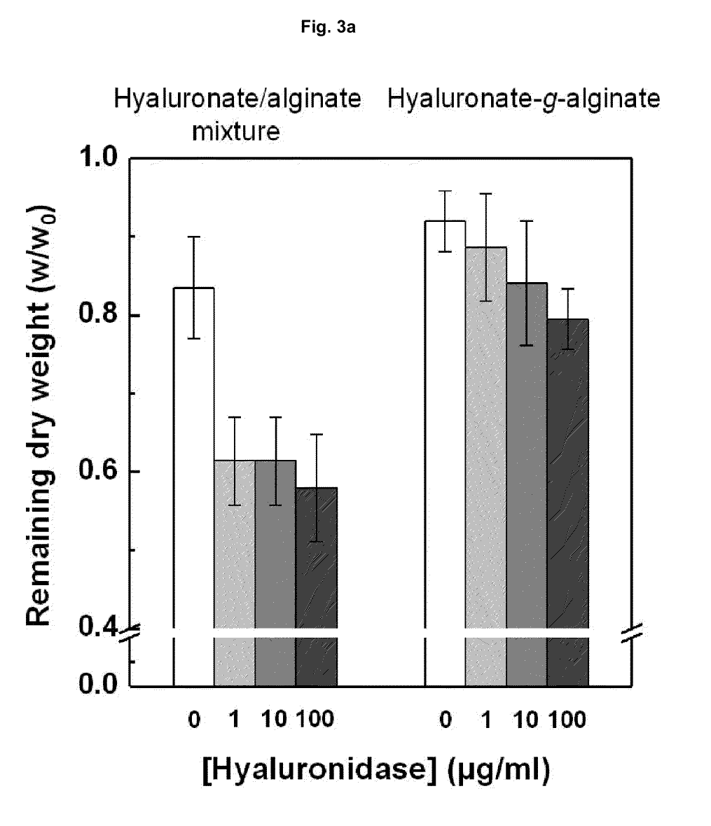

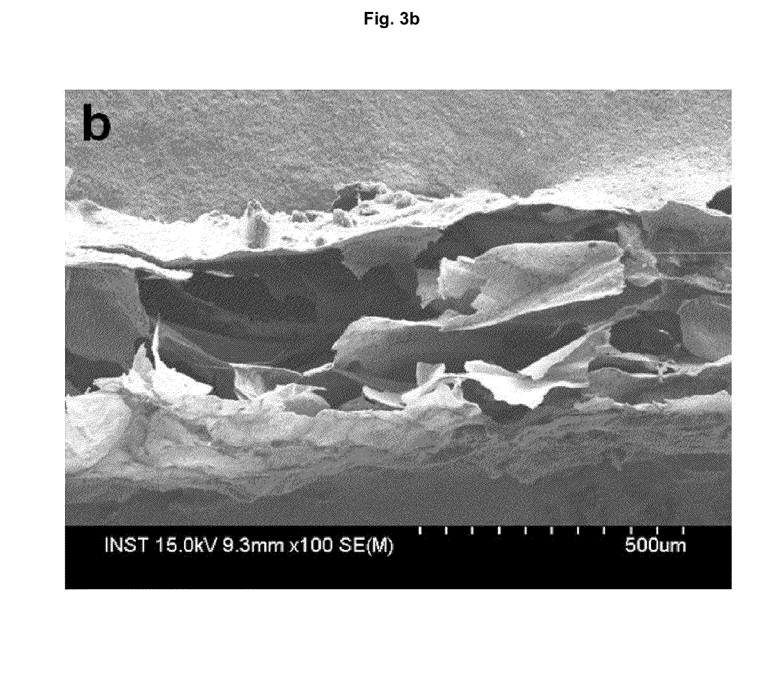

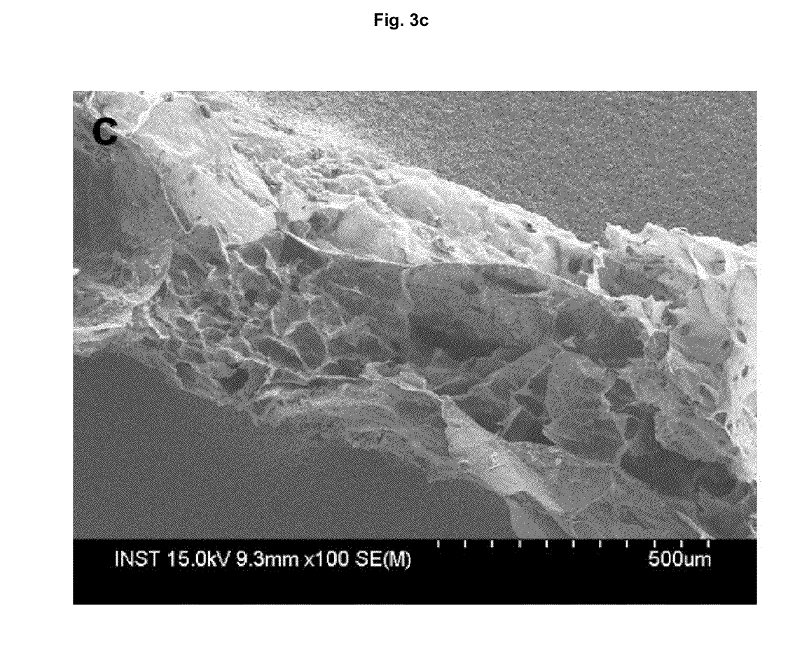

Alginate-grafted hyaluronate gels were treated with hyaluronidase to test stability against enzyme reactions in vitro. At 2 weeks incubation time, slight reductions in gel weights were observed. Gel weights depended on enzyme concentration (FIG. 3a). Hydrogels maintained approximately 80% of their initial weight after treatment with 100 μg/ml of hyaluronidase for 2 weeks compared with control conditions (no enzyme). Hyaluronate-g-alginate gels maintained well-packed, porous structures following enzymatic treatment (FIG. 3c). In contrast, gels prepared from simple mixtures of hyaluronate and alginate exhibited significant reduction of gel weight even in the presence of 1 μg/ml of hyaluronidase during incubation. Approximate weight reduction of 40% was observed, independent of hyaluronidase content. This weight reduction is similar to that expected based on hyaluronate content in the gel. A cross-sectional image of hyaluronate/alginate mixed gel following treatment with hyaluronidase showed substantial gel loss, as caused by enzymatic degradation (FIG. 3b).

Hyaluronate is cleaved by action of hyaluronidase into smaller oligosaccharide fragments [45,46]. However, hyaluronate degrades rapidly in vivo in the absence of chemical modification or cross-linking caused by endogenous hyaluronidase [47]. Fast gel degradation may be unfavorable in regeneration of many types of tissues, as gels should supply 3-D structural environments and maintain their integrity as a scaffold during new ECM development from delivered cells. Hyaluronate/alginate mixed hydrogels do not form complete interpenetrating polymer network structures, because only alginate in mixtures participate in gel formation when calcium ions are added. Hyaluronate in mixed gels was almost entirely degraded by the action of hyaluronidase (FIG. 3). On the other hand, degradation of hyaluronate in hyaluronate-g-alginate gels was restricted by covalent conjugation of alginate chains, leading to slow gel degradation by hyaluronidase. This might make these gels be more appropriate for in vivo applications than mixed gels.

### In Vivo Cartilage Regeneration Using Hyaluronate-g-Alginate Gels

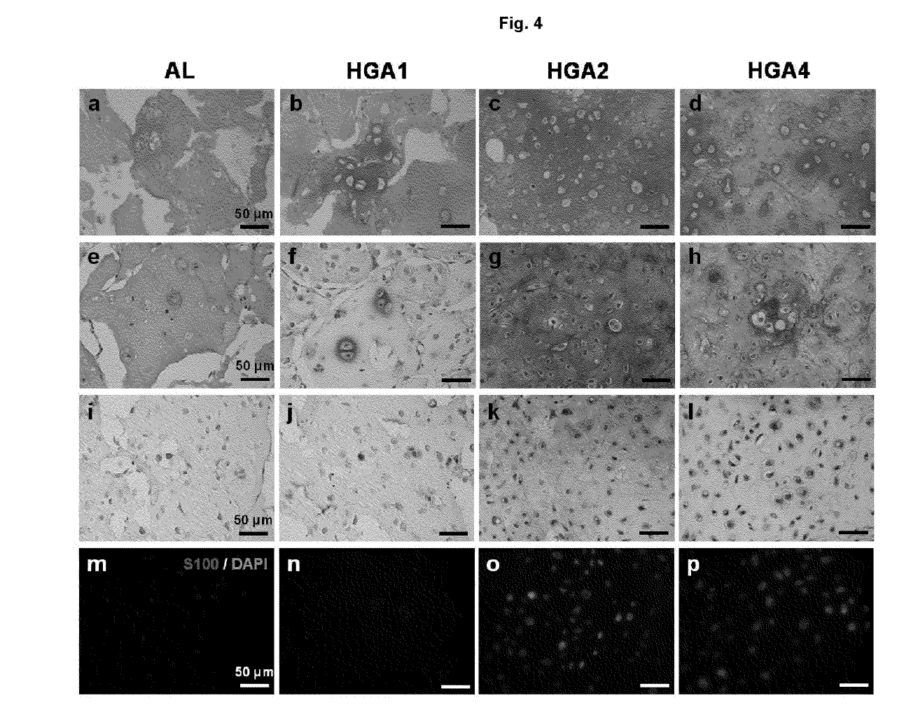

Ionically cross-linkable hyaluronate hydrogels with primary chondrocytes were injected into the dorsal region of mice to verify the efficacy of the gels in cartilage regeneration in vivo. RGD peptides were initially introduced to all hyaluronate-g-alginate gels tested in vivo. RGD-alginate (AL) was also used as a control. Six weeks post-transplantation, tissue volume for each group had not significantly changed as compared with initial injection volumes. This indicates that these gels were able to maintain their 3-D volumetric structures during tissue formation. Tissue sections were stained with Alcian blue (FIG. 4a-d) and Sirius red (FIG. 4e-h) to visualize proteoglycan and collagen, respectively, which probe new ECM formation in regenerated cartilage. ECM formation is essential for transplanted cells with regard to maintenance of viability and functional activity. Homogeneous distributions of lacunae structures were observed in the tissue sections in response to use of HGA2 and HGA4 gels. On the other hand, sparse distributions of lacunae structures were found in AL- and HGA1-treated groups. Interestingly, ECM formation and cartilage lacunae development were more abundant for the HGA2 group, as compared with other groups (FIG. 4c, 4g). HGA4 gels were less efficient than HGA2 gels, likely due to weaker mechanical properties of the HGA4 gels as compared with those of HGA2 gels (FIG. 2d). AL and HGA1 gels were not appropriate for cartilage regeneration in vivo.

Matrillin-1 (FIGS. 4i-l) and S100 proteins (FIG. 4m-p) were visualized with antibodies for immunohistochemical analysis of regenerated tissue sections. Matrillin-1 plays an important role in linking type VI collagen microfibrils and type II collagen fibrils involved in constructing ECM in cartilage tissues [48]. RemarkableMatrillin-1 signals were observed in HGA2 gels (FIG. 4k), as compared with other groups, which is evident in construction of collagen matrices. AL and HGA1 gels did not sufficiently induce protein production (FIG. 4i, j); a finding that was consistent with results obtained from histological analysis. S100 protein is an intracellular calcium binding protein expressed in chondrocytes, adipocytes, Schwann cells and neural cells [49]. S100 protein is expressed at early stages during the chondrogenesis process, and expression fades in conjunction with phenotypic changes in chondrocytes, such as those caused by hypertrophy [50-52]. Positive S100 fluorescent signals were observed in HGA2 and HGA4 groups (FIG. 4o, p), indicating that the transplanted cells maintained their chondrogenic phenotypes in vivo when these hyaluronate-g-alginate gels were injected.

### Quantitative Analysis of GAG Formation and Gene Expression

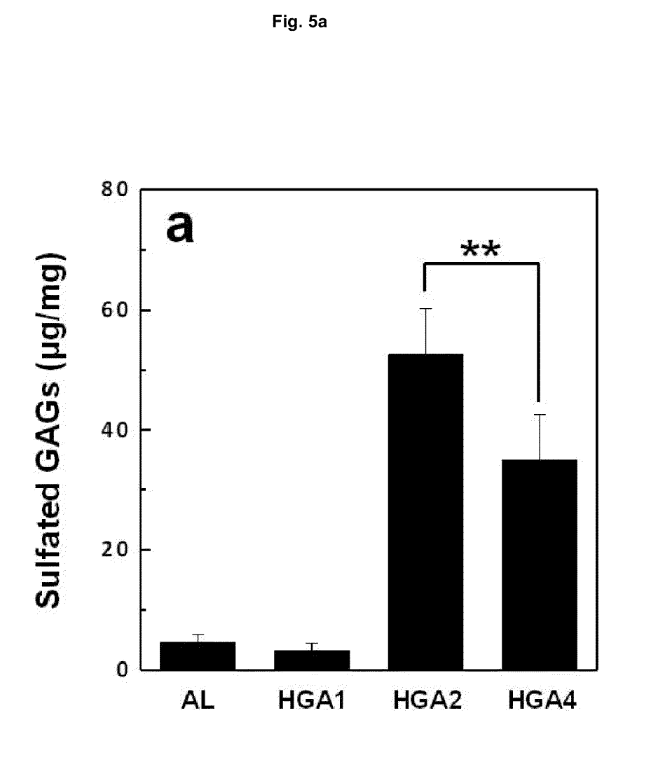

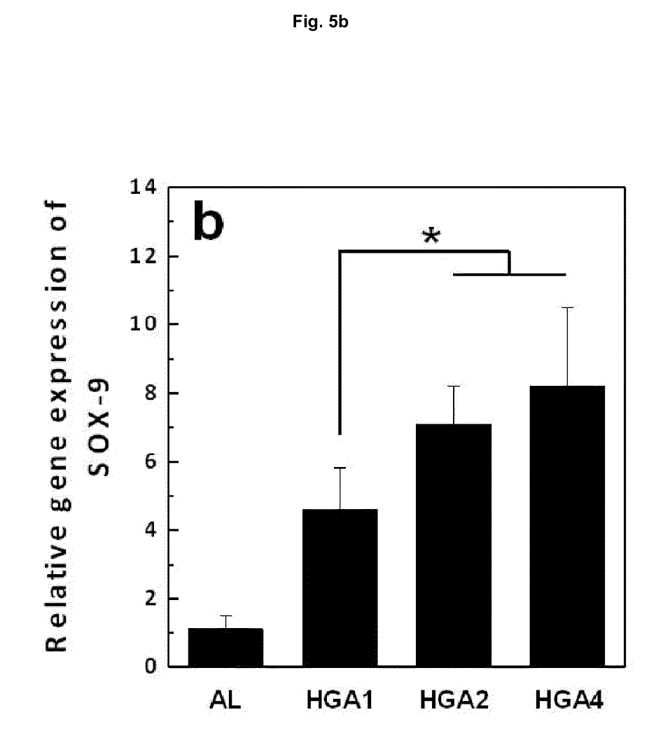

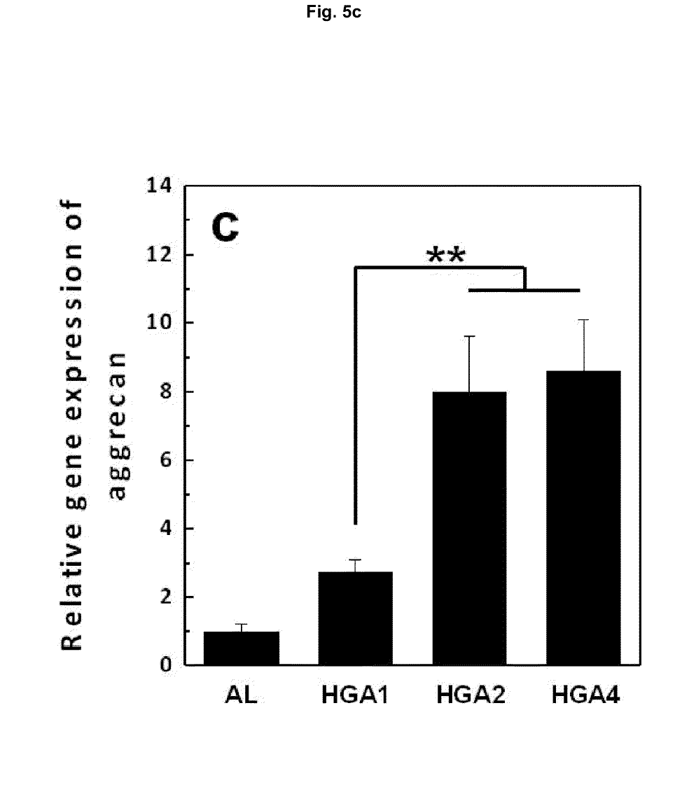

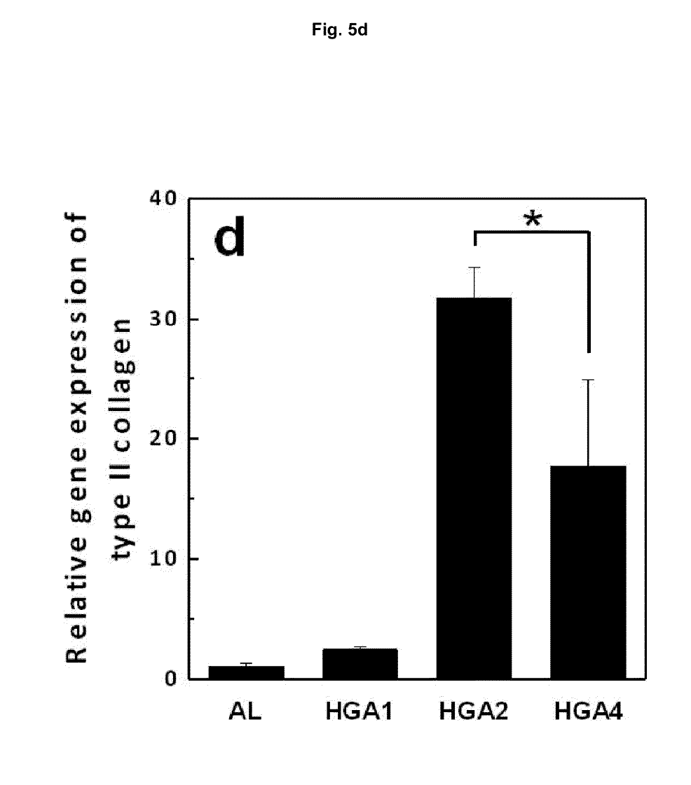

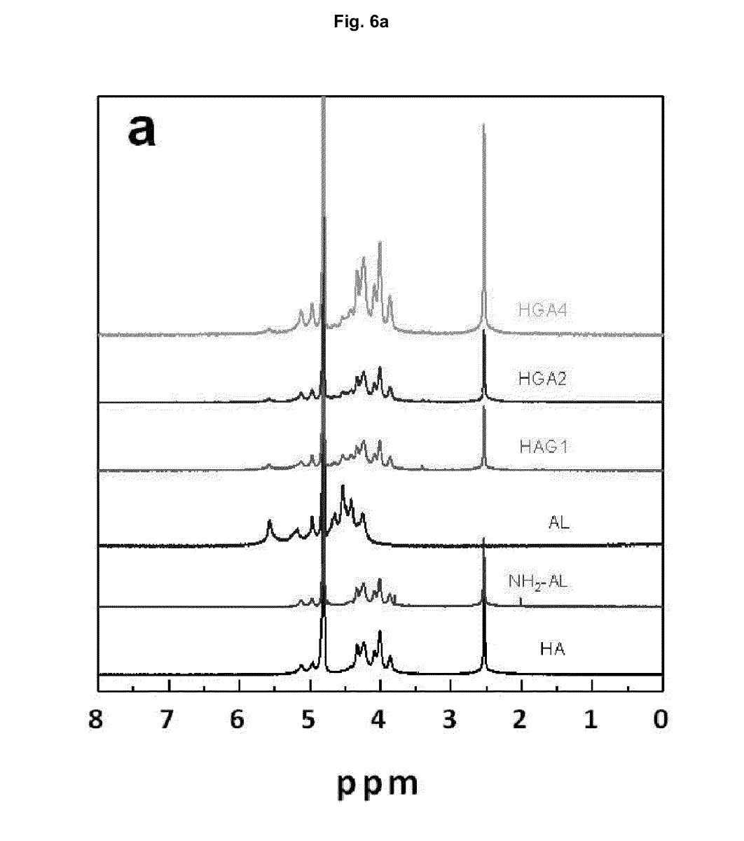

Sulfated glycosaminoglycan (GAG) content was determined and normalized by dried tissue-weight (FIG. 5a). GAGs were rarely formed for the 1HA (3.2±1.1 μg/ml) and AL (4.6±1.3 μg/ml) groups. Significantly more GAGs were secreted when HGA2 gels were used (52.5±7.7 μg/ml) compared with the other groups. Gene expression of typical chondrogenic markers was next investigated (FIG. 5b-d). Gene expression informs cell functionality, which regulates protein production, morphogenesis, cellular differentiation and phenotype. Chondrogenic and ECM-formation processes can be described as related to expression of chondrogenic marker genes such as SOX-9, aggrecan, and type II collagen. SOX-9 is expressed in early development of chondrogenesis, and is critical for chondrocyte differentiation and function [53,54]. Aggrecan and type II collagen are important ECM components of cartilage tissues [55]. In the current study, little expression of chondrogenic marker genes for AL was observed, and enhanced gene expression with hyaluronate was found. Prominent expression of SOX-9, aggrecan and type II collagen was found upon transplantation of primary chondrocytes in HGA2 gels. Relative expression levels of SOX-9, aggrecan, and type II collagen were 7.1±1.1, 8.0±1.6, and 31.8±2.5 times higher than those for the AL group. While HGA4 gels contained more hyaluronate than HGA2 gels, the gels did not enhance in vivo chondrogenic gene expression. In particular, type II collagen expression levels were lower in the HGA4 groups than in the HGA2 groups. These results were consistent with those obtained from histological and immunohistochemical analyses.

In this study, alginate-grafted hyaluronate formed cross-linked structures in the presence of calcium ions, and showed great potential with regard to in vivo cartilage regeneration. In previous studies, we reported that RGD-modified alginate hydrogels could successfully regenerate cartilage tissues in vivo [56,57]. However, approximately 5×10⁷ cells/ml were required for successful cartilage tissue regeneration. This study found that hyaluronate-g-alginate was able to reduce cell concentration (with [cell]=1×10⁷ cells/ml). We also learned that hyaluronate proportion in hyaluronate-g-alginate was an important parameter in cartilage regeneration. The optimal weight ratio between hyaluronate and alginate was found to be two (HGA2), as gel stiffness was found to deteriorate upon addition of hyaluronate (860±70 Pa for HGA2 and 74.6±0.1 Pa for HGA4; FIG. 3d). Thus, harmonization between polymer content and mechanical stiffness should be carefully considered in hopes of obtaining successful in vivo cartilage engineering. Hyaluronate-g-alginate may be a promising candidate for this approach.

Having described a specific embodiment of the present invention, it is to be understood that variants and modifications thereof falling within the spirit of the invention may become apparent to those skilled in this art, and the scope of this invention is to be determined by appended claims and their equivalents.

### REFERENCES

[1] S. K. Hahn, S. J. Kim, M. J. Kim, D. H. Kim, Characterization and in vivo study of sustained-release formulation of human growth hormone using sodium hyaluronate, Pharm. Res. 21 (2004) 1374-1381.
[2] S. J. Kim, S. K. Hahn, M. J. Kim, D. H. Kim, Y. P. Lee, Development of a novel sustained release formulation of recombinant human growth hormone using sodium hyaluronate microparticles, J. Control. Release 104 (2005) 323-335.
[3] J. Elisseeff, K. Anseth, D. Sims, W. McIntosh, M. Randolph, R. Langer, Transdermal photopolymerization for minimally invasive implantation, Proc. Natl. Acad. Sci. U.S.A. 96 (1999) 3104-3107.
[4] C. Chung, M. Beecham, R. L. Mauck, J. A. Burdick, The influence of degradation characteristics of hyaluronic acid hydrogels on in vitro neocartilage formation by mesenchymal stem cells, Biomaterials 30 (2009) 4287-4296.
[5] J. R. E. Fraser, T. C. Laurent, U. B. G. Laurent, Hyaluronan: Its nature, distribution, functions and turnover, J. Intern. Med. 242 (1997) 27-33.
[6] W. S. Toh, E. H. Lee, X. M. Guo, J. K. Y. Chan, C. H. Yeow, A. B. Choo, T. Cao, Cartilage repair using hyaluronan hydrogel-encapsulated human embryonic stem cell-derived chondrogenic cells, Biomaterials 31 (2010) 6968-6680.
[7] Y. Luo, K. R. Kirker, G. D. Prestwich, Cross-linked hyaluronic acid hydrogel films: new biomaterials for drug delivery, J. Control. Release 69 (2000) 169-184.
[8] R. Barbucci, S. Lamponi, A. Borzacchiello, L. Ambrosio, M. Fini, P. Torricelli, R. Giardino, Hyaluronic acid hydrogel in the treatment of osteoarthritis, Biomaterials23 (2002) 4503-4513.
[9] X. Z. Shu, Y. C. Liu, F. Palumbo, G. D. Prestwich, Disulfide-crosslinked hyaluronan-gelatin hydrogel films: a covalent mimic of the extracellular matrix for in vitro cell growth, Biomaterials 24 (2003) 3825-3834.
[10] H. W. Sung, R. N. Huang, L. L. H. Huang, C. C. Tsai, C. T. Chiu, Feasibility study of a natural crosslinking reagent for biological tissue fixation, J. Biomed. Mater. Res. 42 (1998) 560-567.
[11] G. Wei, H. Xu, P. T. Ding, S. M. Li, J. M. Zheng, Thermosetting gels with modulated gelation temperature for ophthalmic use: the rheological and gamma scintigraphic studies, J. Control. Release 83 (2002) 65-74.
[12] M. R. Kim, T. G. Park, Temperature-responsive and degradable hyaluronic acid/Pluronic composite hydrogels for controlled release of human growth hormone, J. Control. Release 80 (2002) 69-77.
[13] S. E. Burke, C. J. Barrett, pH-responsive properties of multilayered poly(L-lysine)/hyaluronic acid surfaces, Biomacromolecules 4 (2003) 1773-1783.
[14] S. J. Kim, C. K. Lee, Y. M. Lee, I. Y. Kim, S. I. Kim, Electrical/pH-sensitive swelling behavior of polyelectrolyte hydrogels prepared with hyaluronic acid-poly(vinyl alcohol) interpenetrating polymer networks, React. Funct. Polym. 55 (2003) 291-298.
[15] C. W. Chung, J. Y. Kang, I. S. Yoon, H. D. Hwang, P. Balakrishnan, H. J. Cho, K. D. Chung, D. H. Kang, D. D. Kim, Interpenetrating polymer network (IPN) scaffolds of sodium hyaluronate and sodium alginate for chondrocyte culture, Colloid Surf. B-Biointerfaces 88 (2011) 711-716.
[16] G. D'Arrigo, C. Di Meo, E. Geissler, T. Coviello, F. Alhaique, P. Matricardi, Hyaluronic acid methacrylate derivatives and calcium alginate interpenetrated hydrogel networks for biomedical applications: physico-chemical characterization and protein release. Colloid Polym. Sci. 290 (2012) 1575-1582.
[17] A. L. Sieminski, C. E. Semino, H. Gong, R. D. Kamm, Primary sequence of ionic self-assembling peptide gels affects endothelial cell adhesion and capillary morphogenesis, J. Biomed. Mater. Res. Part A87A (2008) 494-504.
[18] S. E. Paramonov, H. W. Jun, J. D. Hartgerink, Modulation of peptide-amphiphile nanofibers via phospholipid inclusions, Biomacromolecule s7 (2005) 24-26.
[19] A. Haug, B. Larsen, O. Smidsrod, A study of the constitution of alginic acid by partial acid hydrolysis, Acta Chem. Scand. 20 (1966) 183-190.
[20] E. Frei, R. D. Preston, Configuration of alginic acid in marine brown algae, Nature 196 (1962) 130-134.
[21] J. A. Rowley, G. Madlambayan, D. J. Mooney, Alginate hydrogels as synthetic extracellular matrix materials, Biomaterials 20 (1999) 45-53.
[22] K. Y. Lee, D. J. Mooney, Alginate:Properties and biomedical applications, Prog. Polym. Sci. 37 (2012) 106-126.
[23] E. Alsberg, K. W. Anderson, A. Albeiruti, R. T. Franceschi, D. J. Mooney, Cell-interactive alginate hydrogels for bone tissue engineering, J. Dent. Res. 80 (2001) 2025-2029.
[24] K. Y. Lee, E. Alsberg, S. Hsiong, W. Comisar, J. Linderman, R. Ziff, D. Mooney, Nanoscale adhesion ligand organization regulates osteoblast proliferation and differentiation. Nano Lett. 4 (2004) 1501-1506.
[25] E. B. Hunziker, Articular cartilage repair:basic science and clinical progress. A review of the current status and prospects, Osteoarthr. Cartilage 10 (2002) 432-463.
[26] J. A. Buckwalter, H. J. Mankin, Articular cartilage. part II: degeneration and osteoarthrosis, Repair, regeneration, and transplantation, J. Bone Joint Surg. Am. 79 (1997) 612-632.
[27] K. Y. Lee, D. J. Mooney, Hydrogels for tissue engineering, Chem. Rev. 101 (2001) 1869-1879.
[28] R. Jin, L. S. M. Teixeira, P. J. Dijkstra, M. Karperien, C. A. van Blitterswijk, Z. Y. Zhong, J. Feijen, Injectable chitosan-based hydrogels for cartilage tissue engineering. Biomaterials 30 (2009) 2544-2551.
[29] K. L. Spiller, S. A. Maher, A. M. Lowman, Hydrogels for the repair of articular cartilage defects, Tissue Eng. B17 (2011) 281-299.
[30] J. Elisseeff, Injectable cartilage tissue engineering. Expert Opin. Biol. Ther. 4 (2004) 1849-1859.
[31] C. M. Isacke, H. Yarwood, The hyaluronan receptor, CD44, Int. J. Biochem. Cell Biol. 34 (2002) 718-721.
[32] C. B. Knudson, W. Knudson, Hyaluronan and CD44: modulators of chondrocyte metabolism, Clin. Orthop. Relat. Res. 427 (2004) S152-S162.
[33] C. B. Knudson, Hyaluronan and CD44: Strategic players for cell-matrix interactions during chondrogenesis and matrix assembly, Birth Defects Res. Part C-Embryo Today-Rev. 69 (2003) 174-196.
[34] H. B. Fan, Y. Y. Hu, L. Qin, X. S. Li, H. Wu, R. Lv, Porous gelatin-chondroitin-hyaluronate tri-copolymer scaffold containing microspheres loaded with TGF-beta 1 induces differentiation of mesenchymal stem cells in vivo for enhancing cartilage repair, J. Biomed. Mater. Res. Part A77A (2006)785-794.
[35] S. H. Hsu, S. W. Whu, S. C. Hsieh, C. L. Tsai, D. C. Chen, T. S. Tan, Evaluation of chitosan-alginate-hyaluronate complexes modified by an RGD-containing protein as tissue-engineering scaffolds for cartilage regeneration, Artif. Organs 28 (2004) 693-703.
[36] H. B. Fan, Y. Y. Hu, C. L. Zhang, X. S. Li, R. Lv, L. Qin, R. Zhu, Cartilage regeneration using mesenchymal stem cells and a PLGA-gelatin/chondroitin/hyaluronate hybrid scaffold, Biomaterials 27 (2006) 4573-4580.
[37] J. M. Lohre, L. Baclig, J. Sagartz, S. Guida, K. Thyagarajan, R. Tu, Evaluation of two epoxy ether compounds for biocompatible potential, Artif. Organs 16 (1992) 630-633.
[38] T. Takigawa, Y. Endo, Effects of glutaraldehyde exposure on human health, J. Occup. Health 48 (2006) 75-87.
[39] M. H. Zaman, L. M. Trapani, A. Sieminski, D. MacKellar, H. Y. Gong, R. D. Kamm, A. Wells, D. A. Lauffenburger, P. Matsudaira, Migration of tumor cells in 3D matrices is governed by matrix stiffness along with cell-matrix adhesion and proteolysis, Proc. Natl. Acad. Sci. U.S.A. 103 (2006) 10889-10894.
[40] C. R. Lee, A. J. Grodzinsky, M. Spector M, The effects of cross-linking of collagen-glycosaminoglycan scaffolds on compressive stiffness, chondrocyte-mediated contraction, proliferation and biosynthesis, Biomaterials22 (2001) 3145-3154.
[41] D. E. Discher, P. Janmey, Y. L. Wang, Tissue cells feel and respond to the stiffness of their substrate, Science 310 (2005) 1139-1143.
[42] A. J. Engler, S. Sen, H. L. Sweeney, D. E. Discher, Matrix elasticity directs stem cell lineage specification, Cell 126 (2006) 677-689.
[43] I. Levental, P. C. Georges, P. A. Janmey, Soft biological materials and their impact on cell function, Soft Matter 3 (2007) 299-306.
[44] S. Huang, D. E. Ingber, The structural and mechanical complexity of cell-growth control, Nat. Cell Biol. 1 (1999) E131-E138.
[45] J. Necas, L. Bartosikova, P. Brauner, J. Kolar, Hyaluronic acid (hyaluronan): a review, Vet. Med. 53 (2008) 397-411.
[46] R. Stern, M. J. Jedrzejas, Hyaluronidases: Their Genomics, Structures, and Mechanisms of Action, Chem. Rev. 106 (2006) 818-839.
[47] X. Z. Shu, G. D. Prestwich, Chapter 22 Therapeutic Biomaterials from Chemically Modified Hyaluronan, In:H. G. Garg, C. A. Hales (Eds.), Chemistry and Biology of Hyaluronan, Elsevier, Boston, 2004. pp. 485-504.
[48] C. Wiberg, A. R. Klatt, R. Wagener, M. Paulsson, J. F. Bateman, D. Heinegrd, M. Morgelin, Complexes of matrilin-1 and biglycan or decorin connect collagen VI microfibrils to both collagen II and aggrecan, J. Biol. Chem. 278 (2003) 37698-37704.
[49] T. W. R. Briggs, S. Mahroof, L. A. David, D. J. Flannelly, J. Pringle, M. Bayliss, Histological evaluation of chondral defects after autologous chondrocyte implantation of the knee, J. Bone Joint Surg.-Br. Vol. 85b (2003) 1077-1083.
[50] D. A. Wolff, S. Stevenson, V. M. Goldberg. S-100 protein immunestaining identifies cells expressing a chondrocytic phenotype during articular cartilage repair, J. Orthop. Res. 10 (1992) 49-57.
[51] S. Giovannini, J. Diaz-Romero, T. Aigner, P. Mainil-Varlet, D. Nesic, Population doublings and percentage of S100-positive cells as predictors of in vitro chondrogenicity of expanded human articular chondrocytes, J. Cell Physiol. 222 (2010) 411-420.
[52] C. C. Wang, K. C. Yang, K. H. Lin, Y. L. Liu, H. C. Liu, F. H. Lin, Cartilage regeneration in SCID mice using a highly organized three-dimensional alginate scaffold, Biomaterials33 (2012) 120-127.
[53] H. Akiyama, M. C. Chaboissier, J. F. Martin, A. Schedl, B. de Crombrugghe, The transcription factor Sox9 has essential roles in successive steps of the chondrocyte differentiation pathway and is required for expression of Sox5 and Sox6, Gene Dev. 16 (2002) 2813-2828.
[54] H. Akiyama, Control of chondrogenesis by the transcription factor Sox9, Mod. Rheumatol. 18 (2008) 213-219.
[55] F. Barry, R. E. Boynton, B. S. Liu, J. M. Murphy, Chondrogenic differentiation of mesenchymal stem cells from bone marrow: Differentiation-dependent gene expression of matrix components, Exp. Cell Res. 268 (2001) 189-200.
[56] H. Park, S. W. Kang, B. S. Kim, D. J. Mooney, K. Y. Lee, Shear-reversibly crosslinked alginate hydrogels for tissue engineering, Macromol. Biosci. 9 (2009) 895-901.
[57] H. Park, K. Y. Lee, Facile control of RGD-alginate/hyaluronate hydrogel formation for cartilage regeneration, Carbohyd. Polym. 86 (2011) 1107-1112.

What is claimed is:

1. An ionically cross-linkable alginate-grafted hyaluronate compound containing alginate and hyaluronate, the alginate and the hyaluronate forming a covalent linkage.

2. The compound of claim 1, wherein the alginate has a molecular weight of 10,000 to 1,000,000 g/mol and the hyaluronate has a molecular weight of 10000 to 1000000 g/mole.

3. The compound of claim 1, wherein the weight ratio between the alginate and the hyaluronate is 10:1 to 1:10.

4. The compound of claim 1, wherein the covalent linkage is made via a linker capable of forming covalent linkages with a carboxyl group of the alginate and a carboxyl group of the hyaluronate.

5. The compound of claim 4, wherein the linker is selected from the group consisting of diamine, divinylsulfone, 1,4-butanediol diglycidyl ether (BDDE), glutaraldehyde, carbodiimide, hydroxysuccinimide, imidoester, maleimide, haloacetyl, disulfide hydroazide, and alkoxy amine.

6. The compound of claim 5, wherein the linker is diamine.

7. The compound of claim 1, wherein a cell-adhesive peptide is linked to at least one of the alginate and the hyaluronate.

8. The compound of claim 7, wherein the cell-adhesive peptide is at least one selected from the group consisting of RGD(Arg-Gly-Asp), RGDS(Arg-Gly-Asp-Ser) (SEQ ID NO: 1), RGDC(Arg-Gly-Asp-Cys) (SEQ ID NO: 2), RGDV(Arg-Gly-Asp-Val) (SEQ ID NO: 3), RGES(Arg-Gly-Glu-Ser) (SEQ ID NO: 4), RGDSPASSKP(Arg-Gly-Asp-Ser-Pro-Ala-Ser-Ser-Lys-Pro) (SEQ ID NO: 5), GRGDS(Gly-Arg-Gly-Asp-Ser) (SEQ ID NO: 6), GRADSP(Gly-Arg-Ala-Asp-Ser-Pro) (SEQ ID NO: 7), KGDS(Lys-Gly-Asp-Ser) (SEQ ID NO: 8), GRGDSP(Gly-Arg-Gly-Asp-Ser-Pro) (SEQ ID NO: 9), GRGDTP(Gly-Arg-Gly-Asp-Thr-Pro) (SEQ ID NO: 10), GRGES(Gly-Arg-Gly-Glu-Ser) (SEQ ID NO: 11), GRGDSPC(Gly-Arg-Gly-Asp-Ser-Pro-Cys) (SEQ ID NO: 12), GRGESP(Gly-Arg-Gly-Glu-Ser-Pro) (SEQ ID NO: 13), SDGR(Ser-Asp-Gly-Arg) (SEQ ID NO: 14), YRGDS(Tyr-Arg-Gly-Asp-Ser) (SEQ ID NO: 15), GQQHHLGGAKQAGDV (Gly-Gln-Gln-His-His-Leu-Gly-Gly-Ala-Lys-Gln-Ala-Gly-Asp-Val) (SEQ ID NO: 16), GPR(Gly-Pro-Arg), GHK(Gly-His-Lys), YIGSR(Tyr-Ile-Gly-Ser-Arg) (SEQ ID NO: 17), PDSGR(Pro-Asp-Ser-Gly-Arg) (SEQ ID NO: 18), CDPGYIGSR(Cys-Asp-Pro-Gly-Tyr-Ile-Gly-Ser-Arg) (SEQ ID NO: 19), LCFR(Leu-Cys-Phe-Arg) (SEQ ID NO: 20), EIL(Glu-Ile-Leu), EILDV(Glu-Ile-Leu-Asp-Val) (SEQ ID NO: 21), EILDVPST(Glu-Ile-Leu-Asp-Val-Pro-Ser-Thr) (SEQ ID NO: 22), EILEVPST(Glu-Ile-Leu-Glu-Val-Pro-Ser-Thr) (SEQ ID NO: 23), LDV(Leu-Asp-Val), and LDVPS(Leu-Asp-Val-Pro-Ser) (SEQ ID NO: 24).

9. The compound of claim 8, wherein the cell-adhesive peptide is RGD.

10. A method for cell transplantation, comprising:
preparing a composition comprising the compound of claim 1 and cells for transplantation; and
administering to a subject in need thereof the composition.

11. The method of claim 10, wherein the cells are selected from the group consisting of chondrocytes, myoblasts, hepatocytes, osteoblasts, embryonic stem cells, embryonic germ cells, adult stem cells, mesenchymal stem cells, neural stem cells, endothelial stem cells, hematopoietic stem cells, liver stem cells, cardiac stem cells, pancreatic stem cells, endothelial progenitors, outgrowth endothelial cells, hematopoietic stem cells, human neural stem cells, satellite cells, intestinal epithelial cells, smooth muscle cells, and fibroblasts.

12. A method for cartilage regeneration, comprising administrating to a subject in need thereof a pharmaceutical composition comprising a pharmaceutically effective amount of the compound of claim 1.

13. The method of claim 12, wherein the pharmaceutical composition further comprises primary chondrocytes or stem cells for cartilage regeneration.

14. A method for preventing or treating cartilage-injury disorders or diseases, comprising administrating to a subject in need thereof a pharmaceutical composition comprising a pharmaceutically effective amount of the compound of claim 1.

15. The method of claim 14, wherein the cartilage-injury disorders are selected from the group consisting of osteoarthritis, rheumatoid arthritis, meniscus Injury, costochondritis, relapsing polychondritis and chondrosarcoma.

16. A method for preparing an ionically cross-linkable alginate-grafted hyaluronate compound, the method comprising:
reacting alginate or hyaluronate as a first reactant with a linker to covalently link the linker to a carboxyl group of the alginate or the hyaluronate to obtain a reaction product; and
reacting the reaction product with hyaluronate or alginate as a second reactant, to obtain an alginate-grafted hyaluronate compound in which the alginate is linked to the hyaluronate via the linker.

17. The method of claim 16, wherein the linker in the reaction of alginate or hyaluronate with the linker is used at such an amount as to be linked to all the carboxyl groups of the alginate or hyaluronate as the first reactant.


SEQUENCE LISTING (32 sequences)

SEQ ID NO: 1 - protein, 4 aa, Artificial Sequence
  cell-adhesive peptide
RGDS

SEQ ID NO: 2 - protein, 4 aa, Artificial Sequence
  cell-adhesive peptide
RGDC

SEQ ID NO: 3 - protein, 4 aa, Artificial Sequence
  cell-adhesive peptide
RGDV

SEQ ID NO: 4 - protein, 4 aa, Artificial Sequence
  cell-adhesive peptide
RGES

SEQ ID NO: 5 - protein, 10 aa, Artificial Sequence
  cell-adhesive peptide
RGDSPASSKP

SEQ ID NO: 6 - protein, 5 aa, Artificial Sequence
  cell-adhesive peptide
GRGDS

SEQ ID NO: 7 - protein, 6 aa, Artificial Sequence
  cell-adhesive peptide
GRADSP

SEQ ID NO: 8 - protein, 4 aa, Artificial Sequence
  cell-adhesive peptide
KGDS

SEQ ID NO: 9 - protein, 6 aa, Artificial Sequence
  cell-adhesive peptide
GRGDSP

SEQ ID NO: 10 - protein, 6 aa, Artificial Sequence
  cell-adhesive peptide
GRGDTP

SEQ ID NO: 11 - protein, 5 aa, Artificial Sequence
  cell-adhesive peptide
GRGES

SEQ ID NO: 12 - protein, 7 aa, Artificial Sequence
  cell-adhesive peptide
GRGDSPC

SEQ ID NO: 13 - protein, 6 aa, Artificial Sequence
  cell-adhesive peptide
GRGESP

SEQ ID NO: 14 - protein, 4 aa, Artificial Sequence
  cell-adhesive peptide
SDGR

SEQ ID NO: 15 - protein, 5 aa, Artificial Sequence
  cell-adhesive peptide
YRGDS

SEQ ID NO: 16 - protein, 15 aa, Artificial Sequence
  cell-adhesive peptide
GQQHHLGGAKQAGDV

SEQ ID NO: 17 - protein, 5 aa, Artificial Sequence
  cell-adhesive peptide
YIGSR

SEQ ID NO: 18 - protein, 5 aa, Artificial Sequence
  cell-adhesive peptide
PDSGR

SEQ ID NO: 19 - protein, 9 aa, Artificial Sequence
  cell-adhesive peptide
CDPGYIGSR

SEQ ID NO: 20 - protein, 4 aa, Artificial Sequence
  cell-adhesive peptide
LCFR

SEQ ID NO: 21 - protein, 5 aa, Artificial Sequence
  cell-adhesive peptide
EILDV

SEQ ID NO: 22 - protein, 8 aa, Artificial Sequence
  cell-adhesive peptide
EILDVPST

SEQ ID NO: 23 - protein, 8 aa, Artificial Sequence
  cell-adhesive peptide
EILEVPST

SEQ ID NO: 24 - protein, 5 aa, Artificial Sequence
  cell-adhesive peptide
LDVPS

SEQ ID NO: 25 - DNA, 22 bp, Artificial Sequence
  SOX-9, primer
cttcatgaagatgaccgacgag

SEQ ID NO: 26 - DNA, 22 bp, Artificial Sequence
  SOX-9, primer
ctcttcgctctccttcttgagg

SEQ ID NO: 27 - DNA, 22 bp, Artificial Sequence
  aggrecan, primer
gtgaaaggtgttgtgttccact

SEQ ID NO: 28 - DNA, 21 bp, Artificial Sequence
  aggrecan, primer
tggggtacctgacagtctgat

SEQ ID NO: 29 - DNA, 22 bp, Artificial Sequence
  type II collagen, primer
aagagcggtgactactggatag

SEQ ID NO: 30 - DNA, 21 bp, Artificial Sequence
  type II collagen, primer
tgctgtctccatagctgaagt

SEQ ID NO: 31 - DNA, 22 bp, Artificial Sequence
  GAPDH, primer
gacatcaagaaggtggtgaagc

SEQ ID NO: 32 - DNA, 22 bp, Artificial Sequence
  GAPDH, primer
cttcacaaagtggtcattgagg